(19) United States
(12) Patent Application Publication
(10) Pub. No.: US 2005/0208023 A1
(43) Pub. Date: Sep. 22, 2005

(54) Tumor treating composition and methods

(76) Inventors: Geoffrey Wayne Krissansen, Auckland (NZ); Xueying Sun, Auckland (NZ)

Correspondence Address: FISH & RICHARDSON PC, P.O. BOX 1022, MINNEAPOLIS, MN, 55440-1022, US

(21) Appl. No.: 11/037,395

(22) Filed: Jan. 18, 2005

Related U.S. Application Data
(63) Continuation-in-part of application No. PCT/NZ03/00155, filed on Jul. 18, 2003.

(30) Foreign Application Priority Data
Jul. 19, 2002 (NZ) 520322

(51) Int. Cl. A61K 48/00; C12N 15/86

(52) U.S. Cl. 424/93.2; 435/456; 514/44

(57) ABSTRACT

The invention relates to the treatment of tumors and in particular to methods and compositions for the treatment of solid vascular tumors.

### RELATED APPLICATIONS

This application is a continuation-in-part of International Application Serial No. PCT/NZ03/00155, filed Jul. 18, 2003, which claims priority to New Zealand Application Serial No. 520322, filed Jul. 19, 2002, the contents of which are incorporated herein in their entirety.

### TECHNICAL FIELD

The invention relates to the treatment of tumors and in particular to a composition for the treatment of solid vascular tumors. The invention also relates to compositions and methods of use in such treatments.

### BACKGROUND

Von Hippel-Lindau (VHL) disease is an autosomal dominant familial cancer syndrome that predisposes affected individuals to a variety of highly vascular tumors (1, 2). The most common tumors are hemangioblastomas of the central nervous system, renal cell carcinoma (RCC), and pheochromocytoma. VHL kindreds have germline mutations in the VHL gene, and somatic inactivation or loss of the remaining wild-type VHL allele is linked to tumor formation.

VHL is a tumor suppressor, whose functional inactivation stimulates tumor formation in a variety of ways, in particular by increasing the stability of Hypoxia Inducible Factor-1 (HIF-1) (2, 3). HIF-1 regulates cellular adaption to changes in the oxygen availability by regulating genes involved in angiogenesis, erythropoiesis, energy and iron metabolism, tissue matrix metabolism, and cell survival decisions; which are key factors for tumor growth and survival (4-6). HIF-1 is an αβ heterodimer of which the β subunit is expressed constitutively and is not significantly affected by hypoxia, whereas levels of the α subunit rise markedly with hypoxia, and fall rapidly under normoxic conditions. A 35 amino acid subdomain of the α domain of the 30 kDa von Hippel-Lindau protein (pVHL) binds elongin C, which recruits additional proteins including elongin B, cullin-2, the RING-H2 protein Rbx1/Roc1, and ubiquitin conjugating enzyme E2, to form a ubiquinating complex. The β domain of pVHL binds hypoxia-inducible factor (HIF) α subunits HIF-1α and HIF-2α, targeting them for ubiquitination and proteasomal destruction in a VHL α-domain-dependent manner (7). The binding of HIF-1α subunits to VHL, and their rapid degradation by the VHL ubiquitinating complex under normoxic conditions, is regulated by oxygen and iron-dependent hydroxylation of Pro-564 within HIF-1α (8). Mutation of the α and β domains of VHL either prevents formation of a VHL ubiquitinating complex, and or binding to HIF-1, respectively, leading to stabilization of HIF-1 (3, 7). A hypoxic phenotype results in which increased levels of HIF-1 induce the synthesis of hypoxia-inducible genes such as vascular endothelial growth factor (VEGF), platelet derived growth factor, and glucose transporter-1 (Glut-1), which assist tumor growth by stimulating tumor angiogenesis, and metabolism (9-12).

Reintroduction of wild-type VHL into the VHL-negative tumor RCC in which both VHL alleles are either inactivated or lost, restores VHL-mediated functions, and leads to a loss of tumorigenicity in nude mice (13).

The development and growth of tumors is complex. Despite the positive results in tumor treatment described to date, there would be distinct advantages in providing alternative options which contribute to the available treatments.

### SUMMARY OF THE INVENTION

In one aspect, the invention provides a method of treating tumors in an animal, the method comprising at least the over-expression of VHL in a tumor.

In a further aspect, the invention provides a method of treating small tumors in an animal by engineered over-expression of VHL in the tumor.

In another aspect, the invention provides a method of inhibiting tumor angiogenesis in an animal, the method comprising at least the over-expression of VHL in a tumor.

In another aspect, the invention provides a method of enhancing tumor cell apoptosis in an animal, the method comprising at least the over-expression of VHL in a tumor.

A method of any one of the above mentioned aspects preferably includes the step of administering to the animal an agent adapted to effect over-expression of VHL in a tumor.

Preferably the agent is a vector adapted to express VHL. Preferably, the vector is a nucleic acid vector. Alternatively, the vector is a viral vector comprising nucleic acid in a viral capsid.

Preferably, the agent is one which allows for over-expression of native VHL within the tumor.

Preferably, the agents adapted to effect over-expression of VHL in a tumor are administered intratumorally. Alternatively, the agents are administered systemically.

In another aspect, the invention provides a composition for use in tumor treatment in an animal, the composition comprising an effective amount of an agent adapted to over-express VHL in a tumor, together with one or more suitable carriers.

In another aspect, the invention provides a composition for inhibiting tumor angiogenesis in an animal, the composition comprising an effective amount of an agent adapted to over-express VHL in a tumor, together with one or more suitable carriers.

In another aspect, the invention provides a composition for enhancing tumor cell apoptosis in an animal, the composition comprising an effective amount of an agent adapted to over-express VHL in a tumor, together with one or more suitable carriers.

In a further aspect, the invention provides a method of treating tumors, enhancing tumor cell apoptosis, or inhibiting tumor angiogenesis in an animal, the method comprising at least the administration of an agent which mimics the function of VHL in a tumor (a VHL function mimicking agent).

In another aspect, the invention provides a composition for use in tumor treatment, enhancing tumor cell apoptosis, or inhibiting tumor angiogenesis in animals, the composition comprising an effective amount of an agent adapted to mimic VHL in a tumor, together with one or more suitable carriers.

In another aspect, the invention provides a method of assessing efficacy of over-expressed VHL in animal tumor treatment, the method comprising the steps of engineering over-expression of VHL in tumors of varying sizes in an animal followed by determining the effect this engineered over-expression has on tumor growth.

Preferably a method of the invention involves the administration of a vector adapted to express VHL which is administered in an amount between about 5 μg and 2 mg.

In another aspect, the invention provides a method of treating tumors, enhancing tumor cell apoptosis or inhibiting tumor angiogenesis in an animal, the method comprising at least the step of administering to the animal an effective amount of VHL.

### DRAWINGS

These and other aspects of the present invention, which should be considered in all its novel aspects, will become apparent from the following description, which is given by way of example only, with reference to the accompanying figures, in which:

FIGS. 1A-D Illustrates that intratumoral injection of an expression plasmid encoding VHL downregulates HIF-1α and VEGF in tumors. (A) Immunohistochemistry to analyze the expression of plasmids injected into tumors. Tumors of 0.4 cm diameter were injected with empty pcDNA3 vector (pCDNA3), or an expression plasmid encoding VHL. Tumor sections prepared two days after plasmid injection were stained (brown) for VHL with the rabbit polyclonal anti-VHL antibody FL-181. Magnification, ×100. (B) Overexpression of VHL by intratumoral injection of a VHL expression plasmid downregulates HIF-1α. EL-4 tumors as in (A) were stained with the mouse anti-mouse HIF-1α mAb H1α67. Magnification, ×100. (C) Overexpression of VHL by intratumoral injection of a VHL expression plasmid downregulates VEGF expression. EL-4 tumor sections as in (A), but prepared 4 days after plasmid injection, were stained with the Ab-1 rabbit polyclonal antibody against VEGF. Magnification, ×100. (D) Western blot analysis of homogenates of tumor cells extracted from tumors. Tumor cell homogenates prepared from tumors as in (A) were injected with empty plasmid (lane 1), or VHL (lane 2). They were resolved by SDS-PAGE, and Western blotted with antibodies against VHL and HIF-1α, and VEGF as indicated. (E) Decrease in the percentage of HIF-1α positive-staining cells after injection of a VHL plasmid. The numbers of HIF-1α positive cells in sections (×40 magnification) illustrated in (B) were counted in 10 blindly chosen random fields. n, number of tumors assessed. There was a significant (P<0.01) difference in the numbers of HIF-1α positive cells in sections of tumors injected with empty pCDNA3 plasmid versus tumors injected with VHL plasmid.

FIG. 2 Illustrates that intratumoral injection of an expression plasmid encoding VHL eradicates small tumors. (A) Small EL-4 tumors, approximately 0.1 cm in diameter, were injected at day 0 with expression plasmids encoding VHL, or empty plasmid (Control), and tumor size was recorded for 12 days. Complete tumor regression is denoted by vertical arrows. Mice were euthanased when tumors reached 1 cm in diameter (denoted by stars).

FIGS. 3A-B Illustrates that intratumoral injection of an expression plasmid encoding VHL inhibits tumor angiogenesis. (A) Illustrated are sections prepared from 0.4 cm tumors injected 4 days earlier with empty pcDNA3 vector (pCDNA3), or an expression plasmid encoding VHL. Sections were stained with anti-CD31 antibody MEC13.3 to visualize blood vessels. (B) Measurement of tumor vascularity. Tumor blood vessels stained with the anti-CD31 mAb were counted in 5 blindly chosen random fields to record mean blood vessel counts per section (40× magnification field). n, number of tumors assessed. A significant (P<0.01) difference in mean vessel counts between tumors injected with therapeutic plasmid vectors versus tumors injected with empty pCDNA3 plasmid is donated by stars.

FIGS. 4A-B Illustrates that intratumoral injection of an expression plasmid encoding VHL enhances tumor cell apoptosis. (A) Tumor sections were prepared from 0.4 cm diameter tumors injected 4 days earlier with either empty pCDNA3 vector, or a plasmid encoding VHL. Tumor sections were stained by TUNEL analysis for apoptotic cells (here colored grey). Magnification ×100. (B) TUNEL positive cells were counted to record the apoptosis index (AI) (40× magnification field). n, number of tumors assessed.

### DETAILED DESCRIPTION

The von Hippel-Landau (VHL) tumor suppressor is lost or mutated in patients with VHL cancer syndrome, and in the majority of patients with sporadic renal cell carcinomas (RCCs). VHL binds the α subunits of hypoxia-inducible factor (HIF)-1α, which stimulates tumor angiogenesis and glycolysis, targeting them for ubiquitination and proteasomal destruction. Reintroduction of the VHL gene product (pVHL) inhibits the growth, tumorigenicity, and invasiveness of RCC cells.

The present inventors have now found that intratumoral injection of small (0.1 cm diameter) subcutaneous tumors derived from mouse EL-4 thymic lymphoma cells with a plasmid encoding VHL to over-express VHL in the tumor, resulted in the down-regulation of HIF-1α and vascular endothelial growth factor (VEGF), and inhibited tumor angiogenesis. There was increased tumor cell apoptosis, accompanied by complete eradication of tumors.

In accordance with these findings the invention relates to methods for inhibiting tumor angiogenesis, enhancing tumor cell apoptosis, and ultimately treating tumors in an animal. The methods of the invention are particularly applicable to the treatment of solid vascular tumors, particularly small tumors. The methods may find application in the treatment of hemangioblastomas of the central nervous system (i.e., glioma), renal cell carcinomas, and pheochromocytoma.

As used herein, the term “vascular tumor” should not be taken to imply that such tumors are highly vascular.

As used in relation to the invention, the term “treating” or “treatment” and the like should be taken broadly. They should not be taken to imply that an animal is treated to total recovery. Accordingly, these terms include amelioration of the symptoms or severity of a particular condition or preventing or otherwise reducing the risk of further development of a particular condition.

It should be appreciated that methods of the invention may be applicable to various species of animal, preferably mammals, more preferably humans.

Methods of the invention involve the over-expression of VHL in a tumor. The term “over-expression” should be taken to refer to an increase in VHL expression above the baseline expression level for a particular tumor. “Over-expression” may occur by increasing expression from an endogenous VHL gene (ie that native to the tumor, or to surrounding or adjacent tissue) or via introduction of a VHL-expressing transgene (as will be elucidated further herein).

Accordingly, a method of the invention includes the administration to an animal of an effective amount of an agent adapted to effect over-expression of VHL in a tumor. In addition, the inventors contemplate methods involving the administration of agents adapted to mimic the function of VHL (ie VHL mimetics), or to up-regulate such agents within the tumor.

In the context of the invention an “effective amount” of an agent to be administered to an animal is an amount necessary to at least partly attain a desired response.

In an alternative embodiment, VHL may be administered to increase its levels in a tumor.

In a preferred embodiment of the invention an agent adapted to effect over-expression of VHL in a tumor is a nucleic acid expression vector. Alternatively, the agent is a viral vector comprising a nucleic acid vector contained within a viral capsid.

Persons of general skill in the art to which the invention relates will readily appreciate nucleic acid expression vectors of use in the invention. However, by way of example expression vectors that contain the CMV promoter are highly active in tumors. One explicit example, in the form of pCDNA3 (Invitrogen), is provided herein after under the heading “Methods”.

Such expression vectors may be constructed according to standard techniques and/or manufacturers instructions, having regard to the published nucleic acid sequence of VHL and/or the published amino acid sequence thereof. The nucleic acid and protein sequences for both human and murine VHL are available on publicly accessible databases. For example, human VHL is available on GenBank under the accession number AF010238. The murine VHL sequence information is available on GenBank under the accession number AF513984. A specific example of how such a vector may be constructed is provided herein after under the heading “Methods”.

It should be appreciated that expression vectors of the invention may include various regulatory sequences. For example, they may include tissue specific promoters, inducible or constitutive promoters. Further, they may include enhancers and the like which may aid in increasing expression in certain circumstances. Persons of general skill in the art to which the invention relates will appreciate various regulatory regions which may provide benefit having regard to the tumor to be treated.

In respect of viral vectors, the inventors contemplate the use of such vectors as adeno-associated virus, lentivirus, adenovirus, retroviruses. Viral vectors may be constructed according to standard procedures in the art. The paper Xu, R., Sun, X., Chan, D., Li, H., Tse, L-Y., Xu, S., Xiao, W., Kung, H., Krissansen, G. W., and Fan, S-T. Long-term expression of angiostatin suppresses metastatic liver cancer in mice. Hepatol. 37:1451-60, 2003 provides details of appropriate viral vectors. It will be appreciated that viral vectors will generally be attenuated such that they do not posses their original virulence.

As noted herein before, the effect of VHL on a tumor can also be achieved via the use of agents or factors that stimulate endogenous VHL expression including those that stimulate VHL gene transcription, translation, or protein stability. Examples of such agents include “nonselective” (indomethacin) and COX-2-selective (NS-398) non steroidal anti-inflammatory drugs (NSAIDs)” (14). Skilled persons may appreciate other appropriate agents.

Reagents that mimic the effects of VHL include drugs that interact with VHL effectors, and stimulate a response similar to that of VHL. Peptides and pharmaceutical type reagents based on the VHL protein sequence or structure could be used as VHL mimetics.

It should be appreciated that agents or compounds of use in the invention may be modified to assist their function in vivo for example by reducing their immunogenicity or increasing their lifetime in vivo. Agents may be modified (for example by addition of a carrier peptide or membrane translocating motif (for example Chariot™ peptide; Active Motif, Carlsbad, Calif., USA) as will be known in the art) or formulated with additional agents to allow for their cell permeability and the like. Persons of ordinary skill in the art to which the invention relates will readily appreciate appropriate modifications. However, by way of example, the agents may be PEGylated to increase their lifetime in vivo, based on, e.g., the conjugate technology described in WO 95/32003.

Administration of agents of use in methods of the invention may occur by any means capable of increasing expression of VHL, or a mimetic thereof, in a tumor. Such methods include intratumoral administration and systemic administration. Intratumoral administration may occur via injection (as exemplified herein after) or alternatively direct injection into blood vessels supplying the tumor could occur. Systemic administration may occur by any standard means readily known to the skilled person in the art to which the invention relates having regard to the information herein and to the agent to be administered.

By way of general example, modes of administration may include oral, topical, systemic (eg. transdermal, intranasal, or by suppository), parenteral (eg. intramuscular, subcutaneous, or intravenous injection), intratumoral (eg. by injection, using bollistics); by implantation, and by infusion through such devices as osmotic pumps, transdermal patches, and the like.

Persons of general skill in the art to which the invention relates will be able to readily appreciate the most suitable mode of administration having regard to the therapeutic agent to be used and the tumor to be treated.

While compounds or agents of use in the invention may be administered alone, in general, they will be administered as pharmaceutical compositions in association with at least one or more carriers and/or excipients. Accordingly, compounds may be administered as naked DNAs, or using virus technologies, or as recombinant proteins, peptides, or pharmaceutical compositions, or by other means that any person of ordinary skill in the art would be able to devise.

Compositions may take the form of any standard known dosage form including tablets, pills, capsules, semisolids, powders, sustained release formulation, solutions, suspensions, elixirs, aerosols, liquids for injection, or any other appropriate compositions. Persons of ordinary skill in the art to which the invention relates will readily appreciate the most appropriate dosage form having regard to the nature of the tumor to be treated and the active agents to be used without any undue experimentation. It should be appreciated that one or more active agents described herein may be formulated into a single composition.

As previously mentioned, compounds or agents compatible with this invention might suitably be administered by a sustained-release system. Suitable examples of sustained-release compositions include semi-permeable polymer matrices in the form of shaped articles, e.g., films, or microcapsules. Sustained-release matrices include polylactides (U.S. Pat. No. 3,773,919; EP 58,481), copolymers of L-glutamic acid and gamma-ethyl-L-glutamate, poly(2-hydroxyethyl methacrylate), ethylene vinyl acetate, or poly-D-(−)-3-hydroxybutyric acid (EP 133,988). Sustained-release compositions also include a liposomally entrapped compound. Liposomes containing the compound are prepared by methods known per se: DE 3,218,121; EP 52,322; EP 36,676; EP 88,046; EP 143,949; EP 142,641; Japanese Pat. Appln. 83-118008; U.S. Pat. Nos. 4,485,045 and 4,544,545; and EP 102,324. Ordinarily, the liposomes are of the small (from or about 200 to 800 Angstroms) unilamellar type in which the lipid content is greater than about 30 mole percent cholesterol, the selected proportion being adjusted for the most efficacious therapy. Suitable carriers and/or excipients will be readily appreciated by persons of ordinary skill in the art, having regard to the nature of the agent to be formulated. However, by way of example, suitable liquid carriers, especially for injectable solutions, include water, aqueous saline solution, aqueous dextrose solution, and the like, with isotonic solutions being preferred for intravenous, intraspinal, and intracisternal administration and vehicles such as liposomes being also especially suitable for administration of agents, such as naked nucleic acid vectors to tumors.

In addition to standard diluents, carriers and/or excipients, compositions of the invention may be formulated with additional constituents, or in such a manner, so as to decrease the immunogenicity of an agent to be administered, or help protect its integrity and prevent in vivo degradation, for example. Persons of ordinary skill in the art to which the invention relates will readily appreciate constituents and techniques to this end.

The compositions may be formulated in accordance with standard techniques as may be found in such standard references as Gennaro A R: Remington: The Science and Practice of Pharmacy, 20^{th} ed., Lippincott, Williams & Wilkins, 2000, for example.

As will be appreciated, the dose of an agent or composition administered, the period of administration, and the general administration regime may differ between subjects depending on such variables as the severity of symptoms, the type of tumor to be treated, the mode of administration chosen, type of composition, size of a unit dosage, kind of excipients, the age and/or general health of a subject, and other factors well known to those of ordinary skill in the art.

The issue of what is, or is not, a small tumor would be readily discernible by trial and error, following the surprising findings disclosed herein.

In relation to intratumoral administration of a nucleic acid vector adapted to express VHL, dosages of between about 5 μg and about 2 mg, may be appropriate, however, this is not definitive.

Administration may include a single daily dose or administration of a number of discrete divided doses as may be appropriate. An administration regime may also include administration of one or more of the active agents, or compositions comprising same, as described herein.

The invention will now be further described with reference to the following non-limiting examples.

### EXAMPLES

Methods

Mice and cell lines. Male C57BL/6 mice, 6-8 weeks old, were obtained from the Animal Resource Unit, Faculty of Medicine and Health Science, University of Auckland, Auckland, New Zealand. The EL-4 thymic lymphoma, which is of C57BL/6(H-2^{b}) origin, was purchased from the American Type Culture Collection (Rockville, Md., USA). It was cultured at 37° C. in DMEM medium (Gibco BRL, Grand Island, N.Y., USA), supplemented with 10% foetal calf serum, 50 U/ml penicillin/streptomycin, 2 mM L-glutamine, 1 mM pyruvate.

Expression plasmids. A cDNA fragment encoding full-length (546 bp) mouse VHL was PCR amplified using IMAGE clone 63956 as a template, and the primers 5′-AGG CGG CGG GGG AGC CCG GTC CTG AGG AGA TGG AGG CTG GGC GGC CGC GGC CGG TGC TGC GCT CG-3′ and 5′-ACT CTC AAG GTG CTC TTG GCT CAG TCG CTG TAT GTC CTT CCG CAC ACT TGG GTA G-3′. The resulting PCR product was used as a template for further amplication with the primers 5′-GGG AAT TCC AAT AAT GCC CCG GAA GGC AGC CAG TCC AGA GGA GGC GGC GGG GGA GCC CGG TCC TG-3′ and 5′-GGT CTA GAT CAA GGC TCC TCT TCC AGG TGC TGA CTC TCA AGG TGC TCT TGG CTC A-3′. The PCR product was subcloned into pCDNA3 (Invitrogen). All constructs were verified by DNA sequence analysis.

Gene transfer of expression plasmids in situ and measurement of anti-tumor activity. Purified plasmids were diluted to 1 mg/ml in a solution of 5% glucose in 0.01% Triton X-100, and mixed in a ratio of 1:3 (wt:wt) with DOTAP cationic liposomes (Boehringer Mannheim, Mannheim, Germany), as described previously (15). Tumors were established by injection of 2×10⁵ EL-4 tumor cells into the right flank of mice, and growth determined by measuring two perpendicular diameters. Animals were killed when tumors reached more than 1 cm in diameter, in accord with Animal Ethics Approval (University of Auckland). Once tumors reached either 0.1 cm in diameter, they were injected with 100 μl expression plasmid (100 μg). Empty pCDNA3 vector served as a control reagent. All experiments included 6 mice per group, and each experiment was repeated at least once.

Immunohistochemistry. Tumor cryosections (10 μm) prepared 2 days following injection of plasmids were incubated overnight with either a rabbit polyclonal antibody against a peptide corresponding to N-terminal amino acids 1-181 of VHL (FL-181, Santa Cruz Biotechnology, Inc), a mouse anti-mouse HIF-1α mAb (H1α67, Novus Biologicals, Inc., Littleton, Colo., USA), or a rabbit polyclonal antibody against VEGF (Ab-1, Lab Vision Corporation; Calif., USA). Rabbit antibody-stained sections were subsequently incubated for 30 min with appropriate secondary antibodies (VECTASTAIN Universal Quick kit, Vector Laboratories, Burlingame, Calif.), and developed with Sigma FAST DAB (3,3′-diaminobenzidine tetrahydrochloride) and CoCl₂ enhancer tablets (Sigma). Sections were counterstained with Mayer's hematoxylin. The Vector M.O.M. Immunodetection Kit (Vector Laboratories, Inc. Burlingame, Calif.) was used to detect the mouse anti-HIF-1α mAb. The total number of HIF-1α positive cells in 10 randomly selected fields was counted, and the percentage of positive staining cells was calculated (percentage of positive cells=number of positive cells×100/total number of cells).

Assessment of vascularity. Methodology to determine tumor vascularity has been described previously (16, 17, 18). Briefly, 10 μm frozen tumor sections, prepared 4 days after injection of 0.4 cm diameter tumors with plasmid, were immunostained with the anti-CD31 antibody MEC13.3 (Pharmingen, Calif.). Stained blood vessels were counted in five blindly chosen random fields (0.155 mm²) at 40× magnification, and the mean of the highest three counts was calculated. The concentric circles method (19, 20) was used to assess vascularity, where 5 to 6 tumor sections were analysed for each plasmid-injected tumor.

In situ detection of apoptotic cells. Serial sections of 6 μm thickness were prepared from excised tumors that had been frozen in liquid nitrogen, and stored at −70° C. Terminal deoxynucleotidyl transferase-mediated deoxyuridine triphosphate-digoxigenin nick end labelling (TUNEL) staining of sections was performed using an in situ apoptosis detection kit from Boehringer Mannheim, Germany. Briefly, frozen sections were fixed with 4% paraformaldehyde solution, permeabilized with a solution of 0.1% Triton-X100 and 0.1% sodium citrate, incubated with TUNEL reagent for 60 min at 37° C., and examined by fluorescence microscopy. Adjacent sections were counterstained with haematoxylin and eosin. The total number of apoptotic cells in 10 randomly selected fields was counted. The apoptotic index was calculated as the percentage of positive staining cells, namely AI=number of apoptotic cells×100/total number of nucleated cells.

Western blot analysis. Tumors previously injected with either empty plasmid, or VHL expression plasmids were excised, minced with scissors and homogenized in protein lysate buffer (50 mmol/L Tris pH 7.4, 100 μmol/L EDTA, 0.25 mol/L sucrose, 1% SDS, 1% NP40, 1 μg/ml leupeptin, 1 μg/ml pepstatin A and 100 μmol/L phenylmethylsulfonyl fluoride) at 4° C. using a motor-driven Virtus homogenizer (Virtus, Gardiner, N.Y.). Tumor lysates from each treatment group were pooled, and debris removed by centrifugation at 10,000×g for 10 min at 4° C. Protein samples (100 μg) were resolved on 10% polyacrylamide SDS gels under reducing conditions, and electrophoretically transferred to nitrocellulose Hybond C extra membranes (Amersham Life Science, Buckingham, England). After blocking the membranes with 5% bovine serum albumin in Tween 20/Tris-buffered saline (TTBS; 20 mmol/L Tris, 137 mmol/L NaCl pH 7.6, containing 0.1% Tween-20), blots were incubated with primary antibodies, and subsequently with horseradish peroxidase-conjugated secondary antibodies. They were developed by enhanced chemiluminescence (Amersham International, Buckingham, England), and exposure to x-ray film. Band density was quantified using Scion Image software (Scion Corporation, Frederick, Md.).

Statistical analysis. Results were expressed as mean values+standard deviation (SD). A student's t test was used for evaluating statistical significance, where a value less than 0.05 (P<0.05) denotes statistical significance.

Results

Engineered over-expression of VHL eradicates small tumors. Whether over-expressed VHL might effectively down-regulate HIF-1 pathways, and angiogenesis in tumors already expressing functional VHL has not been able to be predicted with certainty. In tumors, VHL not only has to contend with HIF-1 induced by hypoxia, but potentially also HIF-1 induced in response to tumor-derived factors such as v-src, and insulin-like growth factor-1 receptor (IGF-1R) ligands (IGF-I, IGF-II, and insulin) (21, 22).

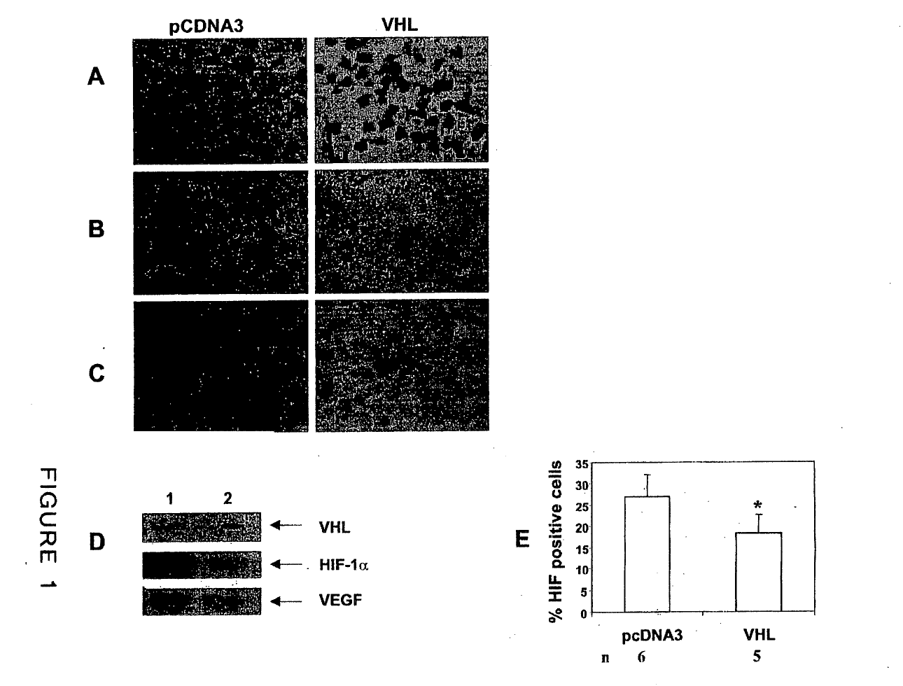

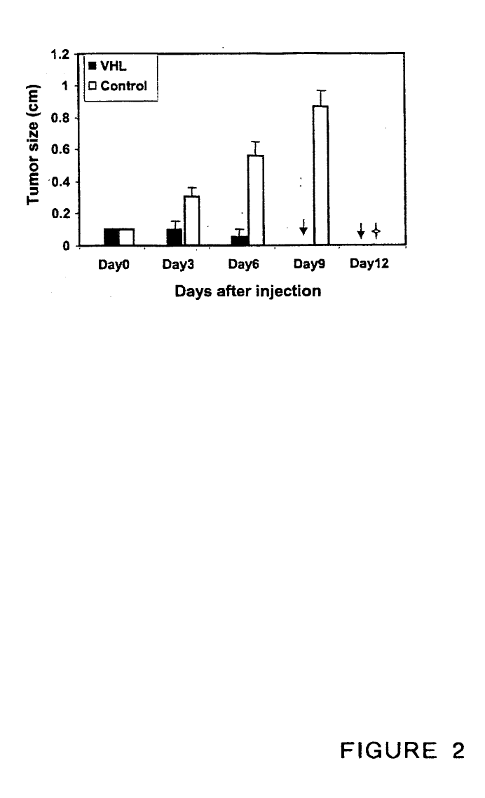

To test the latter notion, small EL-4 tumors, 0.1 cm in diameter, were established in the right flank of C57BL/6 mice, and injected with a DNA/liposome transfection vehicle containing either 100 μg of empty pCDNA3, or VHL-pCDNA3 plasmids. Immunohistochemical analysis of tumor sections, prepared 2d following plasmid injection, revealed that VHL was over-expressed in tumors injected with VHL plasmid, compared to tumors injected with empty plasmid which displayed low levels of endogenous VHL (FIG. 1A). This result was confirmed by Western blot analysis of lysates of tumor cells extracted from tumors, which revealed exogenous VHL was over-expressed 2-fold and was of the expected size of 20 kDa (FIG. 1D). Tumors grew rapidly following injection of empty pCDNA3 plasmid, reaching 1 cm in size within 12 d. In contrast, tumors injected with VHL plasmid rapidly regressed within one week of injection, and completely disappeared (FIG. 2A). Thus, like antisense HIF-1α monotherapy previously reported (16), engineered over-expression of VHL monotherapy is effective against small tumors.

Intratumoral injection of a VHL plasmid down-regulates the expression of HIF-1α and its effector molecule VEGF. In order to understand the mechanisms responsible, in part, for the anti-tumor activity exhibited by exogenous VHL, we examined tumors that had been injected with a VHL plasmid for the levels of HIF-1α, and its effector VEGF. Gene transfer of VHL led to complete downregulation of HIF-1α expression in a proportion (20%) of tumor cells, as revealed by immunohistochemistry (FIGS. 1B and E), and supported by Western blot analysis (FIG. 1D). However, a major proportion of tumor cells appeared to retain some HIF-1α expression (FIGS. 1B and E). Similarly, VHL therapy led to down-regulation of tumoral VEGF expression (FIGS. 1C and D).

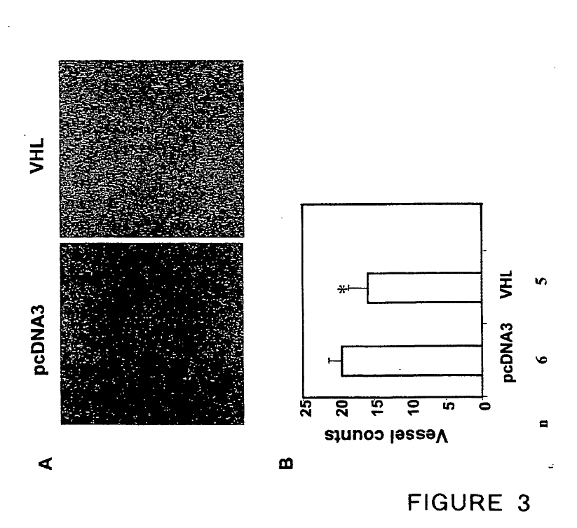

VHL therapy reduces tumor blood vessel density, and increases apoptosis. Injection of a VHL plasmid into tumors inhibited tumor angiogenesis, as evidenced by a statistically significant (p<0.05) reduction in tumor blood vessel density (FIGS. 3A and B), in accord with reductions in the angiogenic factors HIF-1α, and VEGF. The median and 90th centile distances to the nearest CD31-labelled venules from an array of points within tumors treated with VHL plasmid were significantly (both p<0.05) longer than those for tumors treated with empty vector (Table 1). The median and 90th centile distances to the nearest CD31-labelled venules from an array of points within tumors treated with VHL were significantly longer than those for tumors treated with either empty pCDNA3 (P<0.025) plasmid (Table 1).

TABLE 1
Vessel density measured by the concentric circle method

| | |
|---|---|
| Median | 90th Centile |

| Plasmid | | P Value | | P Value |
|---|---|---|---|---|
| pcDNA3 | 18.3 ± 5.2 | | 38.3 ± 5.2 | |
| VHL | 25 ± 4.5 | <0.05 | 43 ± 0 | <0.05 |

The median and 90th centile distance (± SD) to the nearest CD31-labelled venules from an array of points within tumors injected with either empty pcDNA3 or VHL plasmids were determined. P values refer to distances to labelled venules in tumors treated with VHL plasmid versus empty pcDNA3 plasmid.

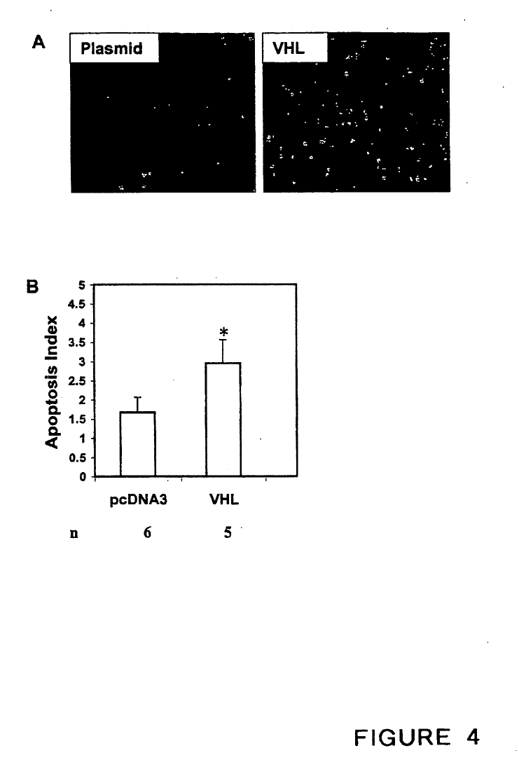

Since tumors were deprived of tumor blood vessels, and survival factors, we examined whether they underwent programmed death as measured by in situ labelling of fragmented DNA using the TUNEL method. A small number of apoptotic cells were detected in tumors injected with empty plasmid (FIG. 4A), whereas tumor apoptosis was almost doubled following injection of VHL plasmid (FIG. 4A, and refer to Apoptosis Index in FIG. 4B). The apoptotic index (AI) for tumors injected with VHL was significantly (P<0.001) different from that of tumors treated with empty pCDNA3 vector.

Discussion

It has been demonstrated for the first time that intratumoral injection of VHL is able to cause complete rejection of small established EL-4 tumors that are not known to carry germline mutations in the VHL gene. VHL therapy caused reductions in the levels of HIF-1α and VEGF in tumors, with a consequent reduction in tumor angiogenesis, and increased tumor cell apoptosis. While tumors did not reappear for three weeks after complete rejection, we cannot discount the possibility that they had regressed to microscopic dormant nodules, rather than being completely eradicated, as occurs with anti-angiogenic therapy employing angiostatin (23) and endostatin (24). The complete and permanent regression of tumors in response to a single injection of VHL gene is similar to that achieved with antisense HIF-1α therapy (16), which is unusual for anti-angiogenic agents where transient suppression of tumor growth is the norm. For antisense HIF-1α therapy, the mechanism involved the NK cell-dependent rejection of tumors (16).

The fact that VHL therapy was very effective at inducing tumor cell apoptosis suggests VHL may have a more predominant role in regulating cell survival, in comparison to its role in regulating HIF-1α expression. VHL appears to exhibit both pro-apoptotic and anti-apoptotic effects, depending on the cellular context. Reintroduction of VHL into VHL-negative RCC cells in vitro provides protection against the cytotoxic effects of serum withdrawal (25), glucose deprivation (26), and UV irradiation (27). It appears to protect renal cells from chemically-induced apoptosis and UV irradiation by inducing Bcl-2 and Bcl-xL expression, and accumulation of cyclin-dependent kinase inhibitors p27 and p21 (28). Since an anti-apoptotic role is counterintuitive to VHL's role as a tumor suppressor, it was argued that loss of VHL may provide selective pressure for tumor cells to override apoptosis (28). The latter results were obtained in in vitro assays, and may not reflect the conditions required for the survival of tumor cells in vivo. Our results suggest that the pro-apoptotic effects of VHL in vivo outweigh its anti-apoptotic effects. It has previously been reported that EL-4 tumors already express the anti-apoptotic factors Bcl-2 (29), and survivin (30), and yet VHL therapy leads to enhanced EL-4 cell apoptosis in vivo. A number of potentially tumor suppressive, and pro-apoptotic properties of VHL have been reported. Thus, VHL interacts with fibronectin, and assists in the assembly of a fibronectin matrix, which can suppress cellular properties associated with malignancy (31). RCC cells engineered to express VHL differentiate and undergo growth arrest when grown to high density on collagen I, whereas VHL negative cells continue to proliferate (32), suggesting that VHL induces differentiation and growth arrest via the integration of cell to cell and cell to matrix signals. Similarly, reintroduction of the VHL gene into mutant RCC cells resulted in growth suppression in vitro, but only when the cells were grown as spheroid cultures, suggesting the effects of VHL are highly dependent on multicellular growth conditions that mimic the basic nature of solid tumors (33). VHL stabilizes actin organization, increases cell adhesion, and inhibits cell motility and invasiveness of tumor cells through focal adhesion formation, and by increasing the expression of tissue inhibitors of metalloproteinases (TIMPS), and decreasing the expression of matrix metalloproteinases 2 and 9 (34). Thus, engineered over-expression of VHL may increase the sensitivity of EL-4 tumors to inhibitory signals from the extracellular matrix.

There are various other mechanisms by which VHL may inhibit tumor growth. VHL interacts in vivo with heteronuclear ribonucleoprotein (hnRNP) A2, an RNA-binding protein that binds a cis-acting instability element in the Glut-1 3′-UTR and protects Glut-1 mRNA from degradation (35). VHL downregulates the expression of hnRNP A2, which leads to a decrease in Glut-1 mRNA. VHL therapy would be expected to inhibit Glut-1 gene transcription, as well as destabilizing already synthesized Glut-1 mRNA, leading to increased inhibition of tumor glycolysis, and decreased tumor metabolism. Reintroduction of the wild-type VHL gene product into RCC cells results in the accumulation of p27, and causes RCC cells to exit the cell cycle and enter G0/quiescence (36). Some tumors such as RCC are dependent on insulin-like growth factor-1 (IGF-1) for tumor growth and invasion. Reintroduction of VHL into RCC cells inhibits IGF-1 receptor signalling via an interaction with protein kinase Cδ, leading to an inhibition of tumor growth and invasion (37).

In both the present report, and a previous publication, we demonstrated that challenging mice with increasing numbers of parental EL-4 cells, causes increased reductions in the generation of anti-tumor CTL, indicating that EL-4 cells are immunosuppressive. One mechanism used by EL-4 cells to evade the immune response involves secretion of TGF-β (38). Thus, the tumorigenicity of EL-4 cells is suppressed by therapy with soluble type II TGF-β receptor (39). Although not tested here, the success of VHL therapy may be due in part to the fact that VHL represses TGF-β1 mRNA and protein levels by decreasing the half-life of TGF-β1 mRNA (40). Reintroduction of VHL into RCC cells was found to neutralize TGF-β activity, causing the regression of established RCC tumors without the development of drug resistance (40). TGF-β1 has proangiogenic effects (41), as evidenced by the finding that targeted disruption of either the TGF-β1 gene or its type II receptor results in defective placental vasculogenesis (42). It appears to synergize with VEGF and bFGF in mediating an angiogenic response (40). VHL also suppresses tumor cell invasion and angiogenesis by upregulating the expression of urokinase-type plasminogen activator mRNA and protein, and conversely downregulating the expression of plasminogen activator 1 mRNA and protein (43). It post-transcriptionally down-regulates the expression platelet derived growth factor (PDGF), and VEGF (44, 45).

Unlike conventional anti-angiogenic agents, VHL therapy appears to inhibit an array of pathways required for tumor growth, and survival. The fact that VHL therapy can cause complete tumor regression is unexpected, and suggests that VHL may, like antisense HIF-1α therapy, expose tumors to the innate immune system which senses danger signals from damaged cells.

While in the foregoing description there has been made reference to specific components or integers of the invention having known equivalents then such equivalents are herein incorporated as if individually set forth.

Although this invention has been described by way of example only and with reference to possible embodiments thereof it is to be understood that modifications or improvements may be made without departing from the scope or spirit of the invention as defined in the appended claims.

The entire disclosures of all applications, patents and publications, cited above and below, if any, are hereby incorporated by reference.

The reference to any prior art in this specification is not, and should not be taken as, an acknowledgment or any form of suggestion that that prior art forms part of the common general knowledge in any country.

Throughout this specification, unless the context requires otherwise, the words “comprise”, “comprising” and the like, are to be construed in an inclusive sense as opposed to an exclusive sense, that is to say, in the sense of “including, but not limited to”.

### REFERENCES

1. Kondo, K., and Kaelin, W. G. 2001. The von Hippel-Landau tumor suppressor gene. Expt. Cell. Res. 264:117-125.
2. McKusick, V. A. 1992. Mendelian Inheritance in Man. Baltimore and London: Johns Hopkins University Press.
3. Krek, W. 2000. VHL takes HIF's breath away. Nature Cell Biol. 2:E1-E3.
4. Blancher, C., and Harris, A. L. 1998. The molecular basis of the hypoxia response pathway: Tumor hypoxia as a therapy target. Cancer Metastasis Rev. 17:187-194, 1998.
5. Wang, G. L., and Semenza, G. L. 1993. General involvement of hypoxia-inducible factor-1 in transcriptional response to hypoxia. Proc. Natl. Acad. Sci. USA 90: 4304-4308.
6. Wang, G. L., Jiang, B-H., Rue, E. A, and Semenza, G. L. 1995. Hypoxia-inducible factor 1 is a basic-helix-loop-helix-PAS heterodimer regulated by cellular O₂ tension. Proc. Natl. Acad. Sci. USA 92:5510-5514.
7. Ohh, M., Park, C. W., Ivan, M., Hoffman, M. A., Kim, T. Y., Huang, L. E, Pavletich, N., Chau, V., and Kaelin, W. G. 2000. Ubiquitination of hypoxia-inducible factor requires direct binding to the beta-domain of the von Hippel-Lindau protein. Nat. Cell. Biol. 2:423-427.
8. Yu, F., White, S. B., Zhao, Q., and Lee, F. S. 2001. HIF-1α binding to VHL is regulated by stimulus-sensitive proline hydroxylation. Proc. Natl. Acad. Sci. USA 98:9630-9635.
9. Jiang B H et al. 1996. Dimerization, DNA binding, and transactivation properties of hypoxia-inducible factor 1. J. Biol. Chem. 271:17771-17778.
10. Ratcliffe, P. J., O'Rourke, J. F., and Maxwell P H, Pugh C W. 1998. Oxygen sensing, hypoxia-inducible factor-1 and the regulation of mammalian gene expression. J. Exp. Biol. 201:1153-1162.
11. Semenza, G. L., Jiang, B. H., Leung, S. W., Passantino, R., Concordet, J. P., Maire, P., and Giallongo, A.1996. Hypoxia response elements in the aldolase A, enolase 1, and lactate dehydrogenase A gene promoters contain essential binding sites for hypoxia-inducible factor 1. J. Biol. Chem. 271:32529-32537.
12. Dachs, G. U., and Stratford, I. J. The molecular response of mammalian cells to hypoxia and the potential for exploitation in cancer therapy. Br. J. Cancer 1996; 74:S126-132.
13. Iliopoulos, O., Kibel, A., Gray, S., and Kaelin, W. G. 1995. Tumor suppression by the human von Hippel-Lindau gene product. Nature Med. 1:822-826.
14. Jones, M. K., Szabo, I. L., Kawanaka, H., Husain, S. S., and Tamawski, A. S. Von Hippel-Lindau tumor suppressor and HIF-1 alpha: new targets of NSAID inhibition of hypoxia-induced angiogenesis. FASEB J. express 2001, 10.1096/fj.01-0589fje.
15. Kanwar, J. R., Berg, R. W., Lehnert, K., and Krissansen G. W. 1999. Taking lessons from dendritic cells: multiple xenogeneic ligands for leukocyte integrins have the potential to stimulate anti-tumor immunity. Gene Therapy 6:1835-1844.
16. Sun, X., Kanwar, J. R., Leung, E., Lehnert, K., Wang, D., and Krissansen, G. W. 2001. Gene transfer of antisense hypoxia inducible factor-1α enhances the therapeutic efficacy of cancer immunotherapy. Gene Therapy 8:638-645.
17. Heather, E. R., Jessica, L., and Randall, S. J. 1998. HIF-1α is required for solid tumor formation and embryonic vascularization. EMBO J. 17:3005-3015.
18. Sun, X., Kanwar, J. R., Leung, E., Lehnert, K., Wang, D., and Krissansen, G. W. 2001. Angiostatin enhances B7.1-mediated cancer immunotherapy independently of effects on vascular endothelial growth factor expression. Cancer Gene Ther. 8:719-727.
19. Kayar, S. R., Archer, P. G., Lechher, A. J., and Banchero, N. 1982. Evaluation of the concentric-circles method for estimating capillary-tissue diffusion distances. Microvascular Res. 24:342-353.
20. Maxwell, P. H., Dachs, G. U., Gleadle, J. M., Nicholls, L. G., Harris, A. L., Stratford, I. J., Hankinson, O., Pugh, C. W., and Ratcliffe, P. J. 1997. Hypoxia-inducible factor-1 modulates gene expression in solid tumors and influences both angiogenesis and tumor growth. Proc. Natl. Acad. Sci. USA 94:8104-8109.
21. Jiang, B-H., Agani, F., Passaniti, A., and Semenza, G. L. 1997. V-src induces expression of hypoxia-inducible factor 1 (HIF-1) and transcription of genes encoding vascular endothelial growth factor and enolase 1: Involvement of HIF-1 in tumor progression. Cancer Res. 57:5328-5335.
22. Feldser, D., Agani, F., Iyer, N. V., Pak, B., Ferreira, G., and Semenza, G. L. 1999. Reciprocal positive regulation of hypoxia-inducible factor 1a and insulin-like growth factor 2. Cancer Res. 59:3915-3918.
23. Cao, Y., O'Reilly, M. S., Marshall, B., Flynn, E., Ji, R. W., and Folkman, J. 1998. Expression of angiostatin cDNA in a murine fibrosarcoma suppresses primary tumor growth and produces long-term dormancy of metastases. J. Clin. Invest. 101:1055-1063.
24. Boehm, T., Folkman, J., Browder, T., and O'Reilly, M. S. 1997. Antiangiogenic therapy of experimental cancer does not induce acquired drug resistance. Nature 390:404-407.
25. Pause, A., Lee, S., Lonergan, K. M., and Klausner, R. D. 1998. The von-Hippel-Lindau tumor suppressor gene is required for cell cycle exit upon serum withdrawal. Proc. Natl. Acad. Sci. USA 95:993-998.
26. Gorospe, M., Egan, J. M., Zbar, B., Lerman, M., Geil, L., Kuzmin, I., and Holbrook, N. J. 1999. Protective function of von Hippel-Lindau protein against impaired protein processing in renal carcinoma cells. Mol. Cell. Biol. 19:1289-1300.
27. Schoenfeld, A. R., Parris, T., Eisenberg, A., Davidowitz, E. J., De Leon, M., Talasazan, F., Devarajan, P., and Burk, R. D. 2000. The von Hippel-Lindau tumor suppressor gene protects cells from UV-mediated apoptosis. Oncogene 19:5851-5857.
28. Devarajan, P., De Leon, M., Talasazan, F., Schoenfeld, A. R., Davidowitz, E. J., and Burk, R. D. 2001. The von Hippel-Lindau gene product inhibits renal cell apoptosis via Bcl-2-dependent pathways. J. Biol. Chem. 276:40599-40605.
29. Grimaite, M., Werner-favre, C., Kindler, V., and Zubler, R. H. 1997. Human naïve B cells cultured with EL-4 T cells mimic a germinal center-related B cell stage before generating plasma cells. Concordant changes in Bcl-2 protein and messenger RNA levels. Eur. J. Immunol. 27:199-205.
30. Kanwar, J. R., Shen, W. P., Berg, R., and Krissansen, G. W. 2001. Effect of survivin antagonists on the growth of established tumors and B7.1 immunogene therapy. J. Natl. Cancer Inst. 93:1541-1552.
31. Ohh, M., Yauch, R. L., Lonergan, K. M., Whaley, J. M., Stemmer-Rachamimov, A. O., Louis, D. N., Gavin, B. J., Kley, N., Kaelin, W. G., and Iliopoulos, O. 1998. The von Hippel-Lindau tumor suppressor protein is required for proper assembly of an extracellular fibronectin matrix. Mol. Cell. 1:959-968.
32. Davidowitz, E. J., Schoenfeld, A. R., and Burk, R. D. 2001. VHL induces renal cell differentiation and growth arrest through integration of cell-cell and cell-extracellular matrix signaling. Mol. Cell Biol. 21:865-874.
33. Lieubeau-Teillet, B., Rak, J., Jothy, S., Iliopoulos, O., Kaelin, W., and Kerbel, R. S. 1998. Von Hippel-Lindau gene-mediated growth suppression and induction of differentiation in renal cell carcinoma cells grown as multicellular tumor spheroids. Cancer Res. 58:4957-4962.
34. Koochekpour, S. et al. 1999. The von Hippel-Lindau tumor suppressor gene inhibits hepatocyte growth factor/scatter factor-induced invasion and branching morphogenesis in renal carcinoma cells. Mol. Cell. Biol. 19:5902-5912.
35. Pioli, P. A., and Rigby, W. F. C. 2001. The von Hippel-Lindau protein interacts with heteronuclear ribonucleoprotein A2 and regulates its expression. J. Biol. Chem. 276:40346-40352.
36. Devarajan, P., De Leon, M., Talasazan, F., Schoenfeld, A. R., Davidowitz, E. J., and Burk, R. D. 2001. The von Hippel-Lindau gene product inhibits renal cell apoptosis via Bcl-2-dependent pathways. J. Biol. Chem. 276:40599-40605.
37. Datta K. Sundberg C. Karumanchi S A. Mukhopadhyay D. 2001. The 104-123 amino acid sequence of the beta-domain of von Hippel-Lindau gene product is sufficient to inhibit renal tumor growth and invasion. Cancer Res. 61:1768-1775.
38. Maeda, H., and Shiraishi, A. 1996. TGF-β contributes to the shift toward Th2-type responses through direct and IL-10-mediated pathways in tumor-bearing mice. J. Immunol. 156:73-78.
39. Won, J., Kim, H., Park, E. J., Hong, Y., Kim, S-J., and Yun, Y. 1999. Tumorigenicity of mouse thymoma is suppressed by soluble type II transforming growth factor β receptor therapy. Cancer Res. 59:1273-1277.
40. Ananth, S., Knebelmann, B., Gruning, W., Dhanabal, M., Walz, G., Stillman, I. E., and Sukhatame, V. P. 1999. Transforming growth factor β1 is a target for the von Hippel-Lindau tumor suppressor and a critical growth factor for clear cell renal carcinoma. Cancer Res. 59:2210-2216.
41. Pepper, M. S., Mandriota, S. J., Vassalli, J. D., Orci, L., and Montesano, R. 1996. Angiogenesis-regulating cytokines: activities and interactions. Curr. Top. Microbiol. Immunol. 213:31-67.
42. Dickson, M. C., Martin, J. S., Cousins, F. M., Kulkarni, A. B., Karlsson, S., and Akhurst, R. J. 1995. Defective haematopoiesis and vasculogenesis in transforming growth factor-1 knockout mice. Development 121:1845-1854.
43. Los, M., Zeamari, S., Foekens, J. A., Gebbink, F. B. G., and Voest, E. E. 1999. Regulation of the urokinase-type plasminogen activator system by the von Hippel-Lindau tumor suppressor gene. Cancer Res. 59:4440-4445.
44. Gnarra, J. R., Zhou, S., Merrill, M. J., Wagner, J. R., Krumm, A., Papavassiliou, E., Oldfield, E. H., Klausner, R. D., and Linehan, W. M. Post-transcriptional regulation of vascular endothelial growth factor mRNA by the product of the VHL tumor suppressor gene. Proc. Natl. Acad. Sci. USA 93:10589-10594.
45. Iliopoulos, O., Levy, A. P., Jiang, C., Kaelin, W. G., and Goldberg, M. A. 1996. Negative regulation of hypoxia-inducible genes by the von Hippel-Lindau protein. Proc. Natl. Acad. Sci. USA 93:10595-10599.

1. A method of treating tumors in an animal, the method comprising at least the over-expression of VHL in a tumor.

2. A method of treating small tumors in an animal by engineered over-expression of VHL in the tumor.

3. A method of inhibiting tumor angiogenesis in an animal, the method comprising at least the over-expression of VHL in a tumor.

4. A method of enhancing tumor cell apoptosis in an animal, the method comprising at least the over-expression of VHL in a tumor.

5. The method of claim 1, wherein it includes the step of administering to the animal an agent adapted to effect over-expression of VHL in a tumor.

6. The method of claim 5, wherein the agent adapted to effect over-expression of VHL in a tumor is a vector adapted to express VHL.

7. The method of claim 6, wherein the vector is a nucleic acid vector.

8. The method of claim 6, wherein the vector is a viral vector comprising nucleic acid in a viral capsid.

9. The method of claim 5, wherein the agent allows for over-expression of native VHL within the tumor.

10. The method of claim 5, wherein the agents adapted to effect over-expression of VHL in a tumor are administered intratumorally.

11. The method of claim 5, wherein the agents are administered systemically.

12. A composition for use in tumor treatment in an animal, the composition comprising an effective amount of an agent adapted to over-express VHL in a tumor, together with one or more suitable carriers and/or excipients.

13. A composition for inhibiting tumor angiogenesis in an animal, the composition comprising an effective amount of an agent adapted to over-express VHL in a tumor, together with one or more suitable carriers and/or excipients.

14. A composition for enhancing tumor cell apoptosis in an animal, the composition comprising an effective amount of an agent adapted to over-express VHL in a tumor, together with one or more suitable carriers and/or excipients.

15. A method of treating tumors, enhancing tumor cell apoptosis, or inhibiting tumor angiogenesis in an animal, the method comprising the administration of an agent which mimics the function of VHL in a tumor.

16. A composition for use in tumor treatment, enhancing tumor cell apoptosis, or inhibiting tumor angiogenesis in animals, the composition comprising an effective amount of an agent adapted to mimic VHL in a tumor, together with one or more suitable carriers and/or excipients.

17. The method of claim 1, wherein the tumor is a small tumor.

18. A method of assessing efficacy of over-expressed VHL in animal tumor treatment, the method comprising the steps of engineering over-expression of VHL in tumors of varying sizes in an animal followed by determining the effect this engineered over-expression has on tumor growth.

19. The use of an agent adapted to effect over-expression of VHL in a tumor in the manufacture of a medicament for use in tumor treatment, enhancing tumor cell apoptosis, or inhibiting tumor angiogenesis in animals.

20. The use of an agent which mimics the function of VHL in a tumor in the manufacture of a medicament for use in tumor treatment, enhancing tumor cell apoptosis, or inhibiting tumor angiogenesis in animals.

21. A method of treating tumors, enhancing tumor cell apoptosis or inhibiting tumor angiogenesis in an animal, the method comprising at least the step of administering to the animal an effective amount of VHL.
